(19) United States
(12) Patent Application Publication
(10) Pub. No.: US 2016/0007944 A1
(43) Pub. Date: Jan. 14, 2016

(54) QUANTIFICATION OF TUMOR ACTIVITY WITH A DUAL-MODALITY ULTRASONIC AND MOLECULAR BREAST IMAGING SYSTEM

(71) Applicant: Michael K. O'CONNOR, Rochester, MN (US)

(72) Inventor: Michael K. O'Connor, Rochester, MN (US)

(21) Appl. No.: 14/771,420

(22) PCT Filed: Feb. 24, 2014
(86) PCT No.: PCT/US14/18009

§ 371 Date: Aug. 28, 2015

Related U.S. Application Data
(60) Provisional application No. 61/771,645, filed on Mar. 1, 2013.

(51) Int. Cl. A61B 6/00; A61B 8/00; A61B 8/08

(52) U.S. Cl. CPC A61B 6/502; A61B 6/4258; A61B 6/4417; A61B 8/0825; A61B 8/403; A61B 8/085; A61B 6/481; A61B 6/469; A61B 6/5247

(57) ABSTRACT

A system and method of quantifying uptake of radionuclide by tumor with the use of an MBI system equipped with an ultrasound unit. The quantification is effectuated based on comparison a first photon count (determined from an MBI image of a region of interest encompassing both the tumor and background tissue of the sample) and a second photon count (determined from an MBI image of a region of a substantially co-extensive region of interest that encompasses only background tissue and is devoid of tumor), and based on the values of depth of tumor and its size determined from the ultrasound imaging data.

### CROSS-REFERENCE TO RELATED PUBLICATIONS

The present patent application claims benefit of and priority from the U.S. Provisional Patent Application No. 61/771,645 titled “Quantification of Tumor Activity with a Dual-Modality Ultrasonic and Molecular Breast Imaging System” (attorney docket 63066.00403) filed on Mar. 1, 2013. The present application is also a continuation-in-part from the U.S. Patent Application s/n 13/701,754 now published as U.S. 2013/0101083, which is a national phase application from PCT/US11/38698 and which claims priority from the U.S. Provisional Patent Application Ser. No. 61/350,644 filed on Jun. 02, 2010. Each of the above-identified patent application(s) is incorporated herein by reference.

### FIELD OF THE INVENTION

The invention relates to systems and methods for breast imaging. More particularly, the invention relates to systems and methods for combined molecular breast imaging and ultrasound imaging.

### BACKGROUND OF THE INVENTION

Breast cancer screening has been recommended for many decades, particularly in women over the age of fifty. The combination of early detection and improved therapy in the U.S. has resulted in a significant reduction in breast cancer mortality, with similar reductions being observed in other countries. Despite the success of screening mammography, however, it is also recognized that mammography is a less than perfect screening method. The limitations of mammography are particularly evident in women with mammographically dense breasts. It has been shown that the sensitivity of mammography decreases with increasing mammographic density, and is less than fifty percent for women with an extremely dense breast pattern on a mammogram.

The reduced sensitivity of mammography with increasing mammographic density is compounded by the fact that increased density is a significant risk factor for breast cancer. Given that a dense breast pattern occurs more frequently in younger women, this factor significantly diminishes the value of mammography in the screening of young women who have a high familial risk of breast cancer.

A second major limitation to screening mammography is in the evaluation of women at high risk of breast cancer. Numerous studies have demonstrated that in women with a high genetic risk of breast cancer, mammography has a sensitivity of between 33-43 percent. Most of these studies have been performed in women with an average age of forty, so part of the explanation for the poor performance of mammography in these studies may be due to the presence of dense breast patterns in a significant percentage of the mammographic images.

A possible solution to the problem of the detection of breast lesions in dense breast tissue is to use ultrasound in such patients. Ultrasound is attractive for supplemental screening because it is widely available, is well-tolerated by patients, and involves no radiation. However, while supplemental ultrasound screening uncovers more breast cancers, it also substantially increases the risk of a false positive cancer finding and unnecessary biopsy. Hence, the use of whole-breast ultrasound as a sole identifier of breast malignancies is questionable. Even in combination with mammography, the two anatomical techniques have significant limitations. It would be of considerable benefit to provide a complementary method that provides functional information about lesions seen on ultrasound. Such a method would significantly reduce the number of false positive cases, and allow the radiologist to evaluate those lesions that demonstrate both a functional and anatomical abnormality.

Over the last several years, a number of nuclear medicine-based technologies have been developed that have application in breast imaging. Included in these are positron emission mammography (“PEM”) and molecular breast imaging (“MBI”). In PEM the breast is compressed between two opposing detectors and the 511 keV gamma rays emitted by a positron emitting radiopharmaceutical, such as F-18 fluoro-deoxyglucose, are detected by coincidence imaging between the two opposing detectors. The PEM images provide an image of glucose utilization by breast tissue and have been shown to be capable of detecting small cancers in the breast. Unlike anatomical techniques such as mammography and ultrasound, PEM is not influenced by dense breast tissue.

The second nuclear medicine-based technique is MBI. This technology employs one or two small gamma cameras. The breast is compressed between a camera and a compression paddle, or between two gamma cameras, and radiation emitted by single-photon radiopharmaceutical(s) (for example, Tc-99m sestamibi) is detected after collimation. MBI is a planar imaging technique without tomographic capability; however, information from two opposing gamma cameras can be used to calculate the depth of a functional abnormality in the MBI images. The MBI system has been shown to have a very high sensitivity, for example in some cases greater than ninety percent, for the detection of lesions smaller than ten millimeters. In addition, it has been found that, in some cases, MBI can detect three times as many cancers as digital and analog mammography in asymptomatic women at increased risk of breast cancer.

Beyond sensitivity differences, technologies that provide functional images of the breast, such as MBI, can detect lesions not visible with conventional mammography. Likewise, certain benign breast conditions may result in a false positive finding on MBI, and this uptake can be readily identified as a benign process from the anatomical information available in ultrasound. Currently it is not practical to fuse anatomical images from ultrasound systems and functional images from MBI.

Ultrasound requires that the patient lie supine and a handheld scanner is then used to scan the breast. MBI is usually performed with the patient seated and the breast lightly compressed between the gamma cameras or camera and paddle. MBI employs light compression forces (for example 10-15 pounds of force) with imaging times in the 5-10 minute range. The imaging procedure is generally considered to be substantially pain-free. Because of the differences in patient orientation used in MBI and ultrasound imaging, the shape of the breast tissue is significantly different between the two modalities and, hence, correlation of an anatomical abnormality with a functional abnormality becomes difficult. Therefore, accurate co-registration of anatomical images from ultrasound and functional information from MBI is not currently possible.

Over the last few years, several entities have worked on the development of whole-breast ultrasound (“WUS”) systems. The main purpose of this development was to reduce the dependence of image quality on the technologist or radiologist, and provide a more reproducible imaging technique. The WUS systems are designed to image the patient in the supine position in a manner comparable to that of conventional breast ultrasound. If the patient is not supine, then the WUS system suffers from a loss in the achievable coverage of the breast tissue. Therefore, while WUS systems provide better coverage in non-supine patient positions than traditional ultrasound imaging, they are still limited in their applicability to combination with imaging modalities that require non-supine patient positions, such as MBI.

It would therefore be desirable to provide an MBI system that would allow the acquisition of both anatomical and functional images of the breast, such images being amenable to co-registration so that accurate and reliable assessments of the presence of cancerous lesions in the breast can be made. Additionally, it would be desirable to provide an MBI system that would also allow for breast biopsies to be performed under the guidance of MBI.

### SUMMARY OF THE INVENTION

The present invention overcomes the aforementioned drawbacks by providing a molecular breast imaging (“MBI)” that is configured for combined MBI and ultrasound imaging or combined MBI and image-guided breast biopsy. Generally, the MBI system includes two opposed gamma ray detectors, in which one of the gamma ray detectors is configured to be moveable with respect to the other such that one gamma ray detector can be moved away from an examination region, thereby permitting ultrasound imaging or image-guided breast biopsy.

Embodiments of the invention provide an MBI system that includes a support structure arranged proximate to an examination region, a first detector head coupled to the support structure to extend along a first portion of the examination region, and a second detector head coupled to the support structure to extend along a second portion of the examination region. The first detector head includes a first gamma ray detector supported by the support structure and extending to define a first imaging plane proximate to the examination region, and a first collimator extending substantially coplanar with the first imaging plane and arranged between examination region and the first detector. Likewise, the second detector head includes a second gamma ray detector supported by the support structure and extending to define a second imaging plane proximate to the examination region, and a second collimator extending substantially coplanar with the second imaging plane and arranged between examination region and the second detector. The MBI system further includes an adjustable coupling arranged between the first detector head and the support structure. The adjustable coupling permits movement of the first detector head between a first position, in which the first detector head is substantially directly opposed to the second detector head, and a second position, in which the first detector head is not substantially directly opposed to the second detector head.

It is another aspect of the invention that the adjustable coupling may be a pivot that defined a rotational axis to permit the first detector head to rotate about the rotational axis to move the first detector head between the first position and the second position.

It is yet another aspect of the invention that the adjustable coupling may be a support arm that is configured to slidably move the first detector head along a direction lying in the first imaging plane to permit the first detector head to move along the direction, thereby moving the first detector head between the first position and the second position.

In a related implementation, the MBI system includes, instead of the second detector head, an ultrasound paddle having an ultrasound transceiver. The ultrasound paddle is attached to the support structure and rotatable about an axis to enable, in operation, compression of the breast between the ultrasound paddle and the first detector head. Such MBI system also includes a control electronic-circuitry unit including data-processing circuitry (such as a computer processor, for example) programmed to (i) acquire, from the first detector head, MBI imaging data representing an MBI image of a region of the breast containing a tumor tissue, the breast having received a radionuclide; (ii) acquire, from the ultrasound transceiver, ultrasound imaging data representing an ultrasound image of the region of the breast to determine depth and size of the tumor tissue; (iii) determine a first photon count corresponding to a first region of interest (ROI) from the MBI image, wherein the first ROI is defined to circumscribe an image of the tumor tissue; and (iv) determine a second photon count corresponding to a second ROI from the MBI image, the second photon count associated with a background portion of the MBI image that is devoid of the image of the tumor tissue. The data-processing circuitry is further configured to calculate a ratio of a first value of radionuclide uptake (by the tumor tissue) to a second value of radionuclide uptake (by a background breast tissue that is devoid of tumor tissue) based on the first and second photon counts, and depth and size of the tumor tissue. The depth and size of the tumor tissue are obtained from the ultrasound imaging data.

Additionally, embodiments of the invention provide a method for quantifying uptake of radionuclide by a breast of a subject with an imaging system that contains a first MBI detector head operably coupled to the support structure and including a first gamma ray detector defining a first imaging plane, a first collimator extending substantially coplanar with the first imaging plane in radiant communication with the first gamma ray detector, and an ultrasound paddle having an ultrasound transceiver juxtaposed therewith and enabled to rotate about an axis. The method includes (i) compressing the breast that has received the radionuclide and that contains a tumor tissue between a first MBI detector head and the ultrasound paddle, and (ii) receiving, from the first MBI detector, first imaging data representing the breast and, from the ultrasound transceiver, second imaging data representing the breast. The method additionally includes a step of identifying a first region of interest (ROI) in an MBI image corresponding to the first imaging data such that the first ROI fully includes an image of the tumor tissue, and a step of identifying a second ROI in the MBI image, which second ROI corresponds to a portion of the MBI image that is devoid of the image of the tumor tissue, and which second RIO is substantially co-extensive with the first ROI. Moreover, the method includes quantitatively determining the photon count representing radionuclide uptake by the tumor tissue as a function of (a) a first photon count received by the first detector head from the first ROI, (b) a second photon count received by the first detector head from the second ROI, and (c) depth and size of the tumor tissue in the breast. In carrying out the method of the invention, the first and second photon counts are associated with the first imaging data, and the depth and size of the tumor tissue are calculated from the second imaging data.

Furthermore, the invention provides an article of manufacture that comprises data-processing electronic circuitry (such as a computer processor, for example) operably cooperated with an imaging system that includes an MBI imaging unit and an ultrasound imaging unit spatially coordinated to compress a breast of a subject therebetween (the breast containing a tumor tissue and having received a radionuclide) and a tangible non-transitory computer-readable medium. This computer-readable medium has computer readable program code thereon which, when loaded on a computer, enables an operation of the data-processing circuitry: (i) to receive first imaging data from the MBI imaging unit and second imaging data from the ultrasound imaging unit; (ii) to determine, from the first imaging data, a first photon count acquired by the MBI imaging unit from a first area of an MBI image that fully contains an image portion representing the tumor tissue and a second photon count acquired by the MBI imaging unit from a second are of the MBI image that is devoid of a portion of the image of the tumor tissue; and (iii) to quantify an uptake of radionuclide by the tumor tissue per unit volume based on the first and second photon counts associated with the first imaging data, and a depth and dimensions of the tumor tissue in the breast that have been calculated from the second imaging data.

In the following description, reference is made to the accompanying drawings which form a part hereof, and in which there is shown by way of illustration of some embodiments of the invention. Such embodiments do not necessarily represent the full scope of the invention, however, and reference is made therefore to the claims and herein for interpreting the scope of the invention.

### BRIEF DESCRIPTION OF THE DRAWINGS

The invention will be more fully understood by referring to the following Detailed Description in conjunction with the generally not-to-scale Drawings, of which:

FIG. 1 is an illustration of an exemplary molecular breast imaging (“MBI”) system for use with the present invention;

FIG. 2 is an exploded view of an example of a gamma detector that forms a part of the MBI system of FIG. 1 and is configured for use with an ultrasound probe or an automated whole-breast ultrasound (“WUS”) system;

FIG. 3 is an elevation view of the example of a gamma detector of FIG. 2;

FIG. 4 is a cross-section of the example of a gamma detector of FIG. 3;

FIG. 5 is an illustration of a configuration of an example of a pair of gantry-mounted opposed gamma detector heads that form a part of an MBI system, in which one gamma detector head is rotatable away from another gamma detector head;

FIG. 6A is an illustration of a configuration of an example of a pair of gantry-mounted opposed gamma detector heads that form a part of an MBI system, in which the opposed gamma detectors are configured to move longitudinally relative to one another;

FIG. 6B is an illustration of the example of a pair of gantry-mounted opposed gamma detector heads of FIG. 6A showing one gamma detector head in a retracted position and another gamma detector head in an extended position;

FIG. 7 is an illustration of an example of a gamma detector head that forms a part of an MBI system configured for use with an ultrasound probe or an automated WUS system;

FIG. 8A is an MBI image of a breast tissue containing tumor; and

FIG. 8B is a flow-chart presenting an example of a method for quantification of an uptake of radionuclide by the tumor in the breast based on depth and size of the tumor determined from data received by the ultrasound probe of FIG. 7 in accordance with an embodiment of the invention.

### DETAILED DESCRIPTION OF THE INVENTION

Current schemes for radio-guided breast biopsy are limited in their ability to incorporate additional information from x-ray or ultrasound imaging. Additionally, such schemed only provide a mechanism to localize a lesion and to perform biopsy, but do not provide any supplementary information about the lesion. Thus, the schemes developed for positron emission mammography (“PEM”) and breast specific gamma imaging (“BSGI”) only provide a conventional imaging scheme to localize a lesion, and neither of these systems incorporates any innovative features to enhance or accelerate the biopsy process. With typical imaging times for both PEM and BSGI at around ten minutes per view, the biopsy process can become a lengthy procedure depending on how many images need to be acquired to confirm correct placement of the biopsy needle.

The MBI system of the present invention provides anatomical correlation information, which is very helpful for aiding a clinician in the decision making process. In breast imaging, the variability in breast position between different modalities can make it difficult to correlate findings across these modalities, particularly in complex cases where both benign and malignant tissue regions may be present. It is contemplated that the ability to co-register an area of increased uptake on an image obtained with MBI with the corresponding area on an ultrasound image will enable physicians to better determine the nature of the lesion and the appropriate course of action.

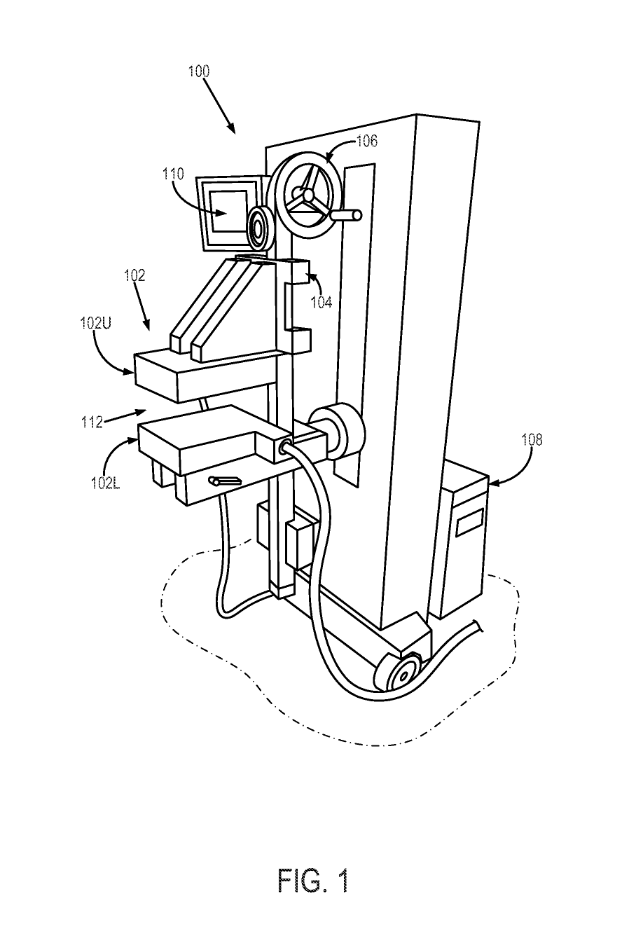

Referring to FIG. 1, a molecular breast imaging (“MBI”) system 100 includes two opposing cadmium zinc telluride (“CZT”) detectors, shown as detector heads 102. In particular, the detector heads 102 include an upper detector head 102U and a lower detector head 102L.

Examples of MBI systems and methods for their use are described, for example, in co-pending U.S. patent application Ser. No. 12/515,369, entitled “System and Method for Quantitative Molecular Breast Imaging,” which is herein incorporated by reference in its entirety. Each detector head 102U, 102L may be, for example, 20-24 centimeters (“cm”) by 16-20 cm in size and mounted on a modified upright type mammographic gantry 104. In one configuration, the detector heads 102 are Lumagem® 3200S high-performance, solid-state cameras from Gamma Medica-Ideas, Inc., having a pixel size of 1.6 millimeters (“mm”) (Lumagem® is a trademark of Gamma Medica-Ideas, Inc., Northridge, Calif.).

The relative position of the detector heads 102 can be adjusted using a user control 106. Specifically, the detector head assemblies 102 are, preferably, designed to serve as a compression mechanism. Accordingly, this system configuration reduces the maximum distance between any lesion in the breast and either detector head 102 to one-half of the total breast thickness, potentially increasing detection of small lesions without additional imaging time or dose. The MBI system 100 includes data-processing circuitry (such as a processor) 108 for processing the signals acquired by the detector heads 102 to produce an image, which may be displayed on an associated display 100.

In general, the detector heads 102U, 102L are arranged so as to form an examination region 112 there between. The examination region 112 is defined with respect to a first imaging plane and a second imaging plane. The first imaging plane is defined, for example, by the extension of the upper detector head 102U along the examination region 112, and the second imaging plane is defined, for example, by the extension of the lower detector head 102L along the examination region 112.

One of the detector heads 102 of an MBI system can be configured to provide an MBI system amenable to combined MBI and ultrasound imaging, or combined MBI and breast biopsy. In other configurations, one of the detector heads 102 can be replaced with an ultrasound probe or a wholebreast US imaging apparatus particularly configured for use with the MBI system. In general, configurations of an MBI system that are amenable for combined MBI and ultrasound imaging, or combined MBI and breast biopsy, include detector heads 102 that can be moved relative to one another such that the breast becomes accessible for an ultrasound imaging system or biopsy device. Thus, in general, the MBI system may include a first detector head that is configured to move between a first position and a second position relative to a second detector head. Generally, such a first position would be one in which the two detector heads are opposed and configured for MBI, and such a second position would be one in which the two detector heads are no longer opposed, thereby providing access to the breast for ultrasonic imaging or biopsy. For example, the upper detector head 102U may be configured to rotate away from the lower detector head 102L, or, the detector heads 102 may be configured to move longitudinally relative to one another.

In general, such a system will provide a more complete imaging solution for women with dense breast tissue where the sensitivity of mammography is know to be limited, and will do so in a cost-effective manner that should permit its widespread adoption into clinical practice.

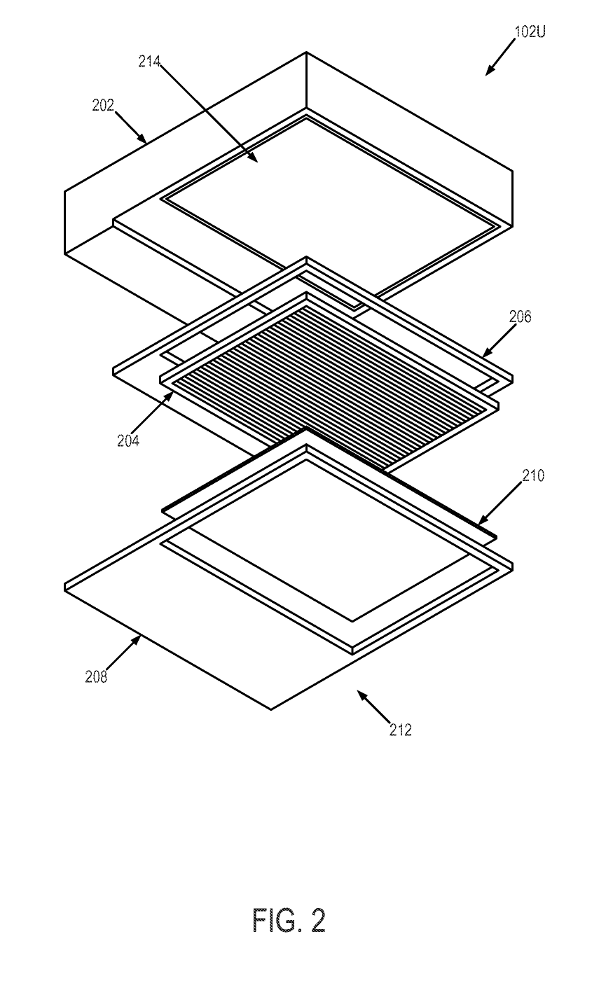

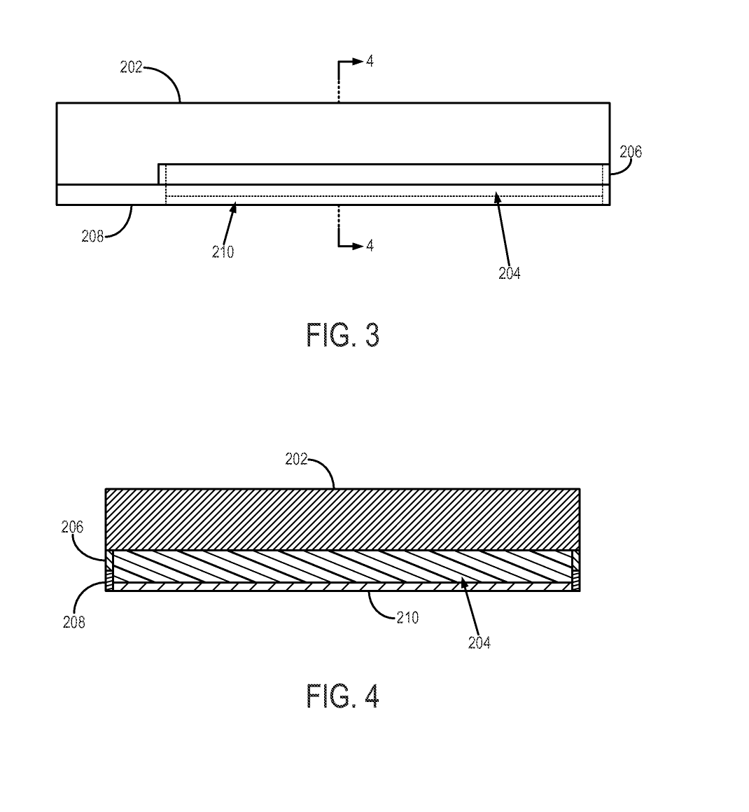

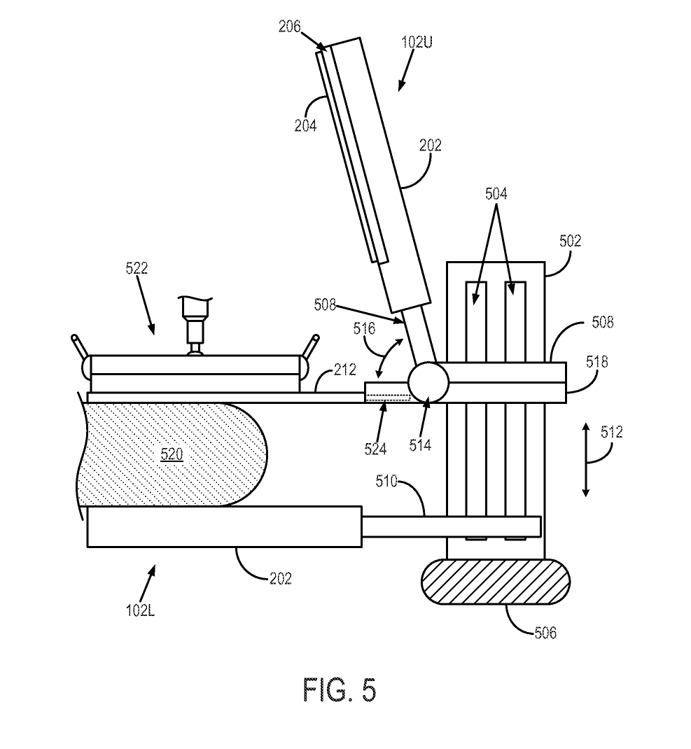

An example of a detector head 102, such as an upper detector head 102U, that is configured for combined MBI and ultrasound imaging, or that is amenable for MBI guided breast biopsy, is illustrated in FIGS. 2-4, to which reference is now made. The detector head 102 includes a gamma ray detector 202, a collimator 204, and an inner collimator frame 206. The gamma ray detector 202 and collimator 204 are arranged such that the collimator 204 is substantially parallel to the imaging plane defined by the detector head 102. The detector head is positioned over an outer collimator frame 208 and an acoustic coupling plate 210. Together, the outer collimator frame 208 and acoustic coupling plate 210 may be referred to as a compression plate 212. Other configurations of a compression plate 212 may include only the acoustic coupling plate 210, an acoustic coupling plate 210 coupled to one or more support members, or a biopsy grid plate coupled to an outer frame, such as the outer collimator frame 208. The inner collimator frame 206 is sized to be received by a recessed portion of the detector head 102. The inner collimator frame 206 is further sized on its inner extent to receive the collimator 204, such that the collimator is positioned in alignment with a detector array 214 formed on a surface of the gamma ray detector 202. By way of example, the detector array 214 may be composed of cadmium zinc telluride (“CZT”) detector elements. The outer collimator frame 208 is sized on its inner extent to receive the acoustic coupling plate 210 and the collimator 204, such that the collimator 204 comes into contact with the acoustic coupling plate 210. The acoustic coupling plate 210 is positioned within the outer collimator frame 208 such that the acoustic coupling plate 210 forms a substantially flush surface with the outer collimator frame 208, thereby providing a contact surface for receiving and compressing a portion of a subject under examination, such as a portion of the subject's breast.

The acoustic coupling plate 210 is composed of a material with low attenuation, and is preferably composed of a material with similar ultrasonic reflective properties as soft tissue. Exemplary materials include nylon and latex. The acoustic coupling plate 210 may also be constructed so as to permit the passage of a biopsy needle through the acoustic coupling plate 210 and into the breast. For example, a nylon mesh can be employed and manufactured with a hole grid to allow a needle to be passed through for biopsy. Additionally, the acoustic coupling plate 210 permits the breast to be retained in a compressed position prior to refraction of the detector head 102.

In ultrasound imaging applications, the precise location of a lesion imaged by the lower detector head 102L can be used to position an ultrasonic probe and permit co-registration of the MBI and ultrasonic images. This may be facilitated by marking the acoustic coupling plate 210, for example, with a grid pattern that can be labeled to match locations on the MBI images. The location of the lesion identified from an MBI image can also be entered into the ultrasound system and an electronic mark on the ultrasound image used to direct and confirm co-registration of the MBI and ultrasound information.

For MBI-guided biopsy applications, the acoustic coupling plate 210 may be replaced by a rigid frame that accommodates a biopsy grid plate, through which a biopsy may be performed. Exemplary biopsy frames are designed to accommodate disposable biopsy grid plates, such as those used in commonly available biopsy devices including the Mammotome Biopsy System (Mammotome, Cincinnati, Ohio) and the ATEC system (Hologic, Inc., Bedford, Mass.).

Current MBI gantry designs work adequately for conventional breast imaging, but do not easily accommodate the integration of an MBI system with another imaging modality, or the introduction of a biopsy device. Thus, the provided MBI system is designed to include detector heads 102 that can be moved so as to provide access to the subject's breast, whether for ultrasound imaging or biopsy.

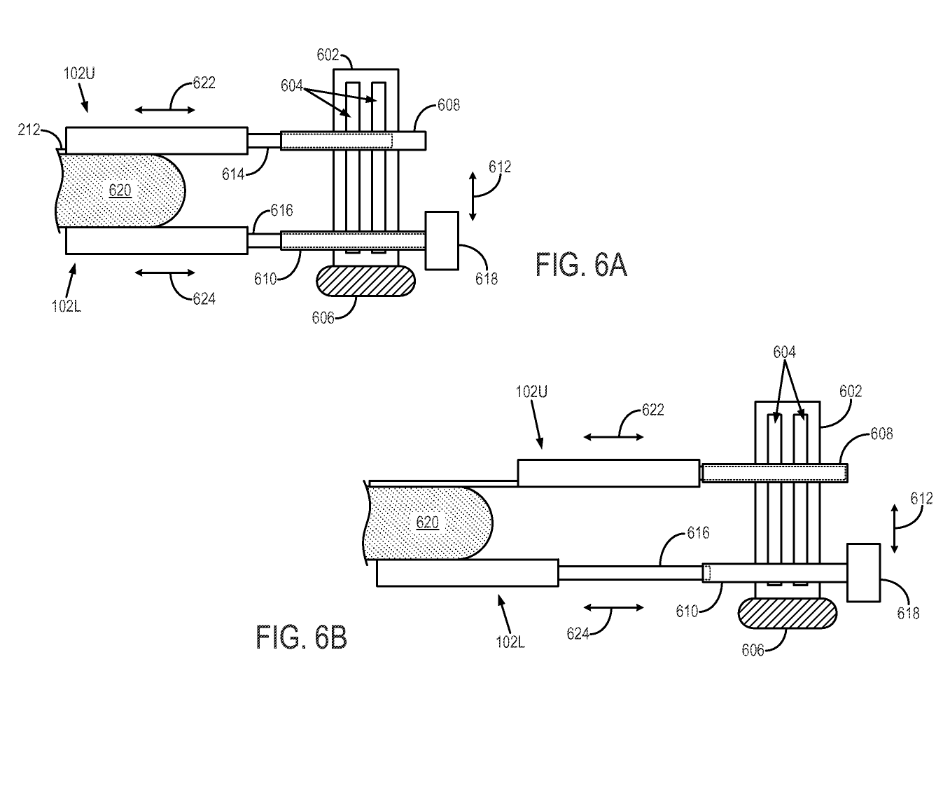

Referring now to FIG. 5, an exemplary configuration of an MBI system having a gantry-mounted, hinged upper detector head 102U that can be rotated away from the lower detector head 102L so as to provide access to the subject's breast for ultrasound imaging or biopsy is illustrated. The MBI system 500 includes a support structure 502 having formed therein one or more tracks 504. The support structure 502 is coupled to a gantry 506 that allows for the MBI system 500 to be positioned about the patient in a number of different orientations. Coupled to the tracks 504 are an upper support arm 508 and a lower support arm 510. The upper detector head 102U is coupled to the upper support arm 508, and the lower detector head 102L is coupled to the lower support arm 510. A motor (not shown in FIG. 5) drives the upper support arm 508 so that the upper detector head 102U is moved along a direction 512 towards or away from the lower detector head 102L. The upper support arm 508 includes a hinge 514 that allows the upper detector head 102U to be rotated away from the lower detector head 102L about a rotational axis of the hinge 514 and along a rotation direction 516. The upper support arm 508 may be coupled to an additional compression support arm 518. When the upper support arm 508 and the compression support arm 518 are together, the compression support arm 518 is used to achieve breast compression with the upper detector head 102U.

The aforementioned hinge 514 is but one example of an adjustable coupling between one detector head 102, such as the upper detector head 102U, and the support structure 502. Generally, this adjustable coupling permits movement of the coupled detector head with respect to the other detector head. More generally, the adjustable coupling permits movement of one detector head between a first position and a second position relative to the other detector head. For example, the first position may be one in which the two detector heads are substantially directly opposed, and the second position may be one in which the two detector heads are not substantially directly opposed to each other. For the configuration illustrated in FIG. 5, in the first position the imaging planes defined by the two detector heads are substantially parallel, and in the second position the imaging plane defined by the upper detector head 102U would be angled away from the imaging plane defined by the lower detector head 102L. In one example, the imaging plane defined by the upper detector head 102U would be rotated such that it is substantially perpendicular to the imaging plane defined by the lower detector head 102L. It should also be appreciated that the hinge 514 that forms an adjustable coupling may also be configured so that the axis of rotation is perpendicular to the imaging plane defined by the coupled detector.

Functional imaging of the breast is performed using the aforementioned MBI system, which permits a calculation of an in-plane location of a lesion in the breast, as well as its depth and relative uptake of an administered radionuclide. Following completion of the MBI acquisition, the upper detector head 102U may be withdrawn, leaving the compression plate 212 in place. By rotating the gamma camera in the upper detector head 102U, the compression plate 212 remains in physical contact with the subject's breast 520. This process allows the maintenance of constant compression of the breast 520 between the lower detector head 102L and the compression plate 212 while the MBI system converts from a molecular imaging mode to an ultrasound imaging or biopsy mode. Moreover, this constant compression of the breast 520 mitigates subject movement while switching between operational modes.

After the upper detector head 102U is rotated away from the breast 520, an ultrasound system 522 may replace the upper MBI detector to either perform a sweep across the breast to obtain three-dimensional images of the breast tissue, or to obtain high resolution images of an area of concern identified on the MBI images. While a wholebreast US system is illustrated in FIG. 5, a conventional hand-held ultrasound transducer may also be used. Upon completion of both the molecular and ultrasound imaging acquisitions, the MBI and ultrasound images are co-registered. Using the MBI system configuration illustrated in FIG. 5, functional and anatomical information are obtained sequentially from the two imaging modalities. As a result, some motion or movement of the breast may occur between the two imaging processes; however, these errors can be addressed during co-registration of the images.

This MBI system configuration is benefited in that the location of a lesion not visible on conventional ultrasound can be determined and indicated on the MBI image, and may also be identifiable from enhanced ultrasonic techniques, such as elastography, thereby permitting ultrasound-guided biopsies if desired. In a high percentage of cases, for example greater than eighty percent, a lesion can be seen on just the lower MBI detector; thus, during ultrasound imaging, information on the location of the lesion can be updated on the ultrasound system to confirm that the location of a lesion has not shifted in the conversion from MBI to ultrasound imaging modes, which can also be useful for addressing inter-imaging motion errors.

The gantry 506 may be designed to be compatible with a biopsy table, such as the DBI Table (Medical Positioning Inc., Kansas city, Missouri). Such a table allows the patient to lie on her side with the superior breast positioned in the MBI system. The back support and patient position minimizes motion and enables better access into the axillary tail and posterior-lateral breast.

Depending on the configuration of the upper support arm 508 and compression support arm 518, the compression plate 212 can be configured to attach to the compression support arm 518, for example by “plugging” the compression plate 212 into appropriately sized holes in the compression support arm 518, as illustrated at arrow 524 in FIG. 5.

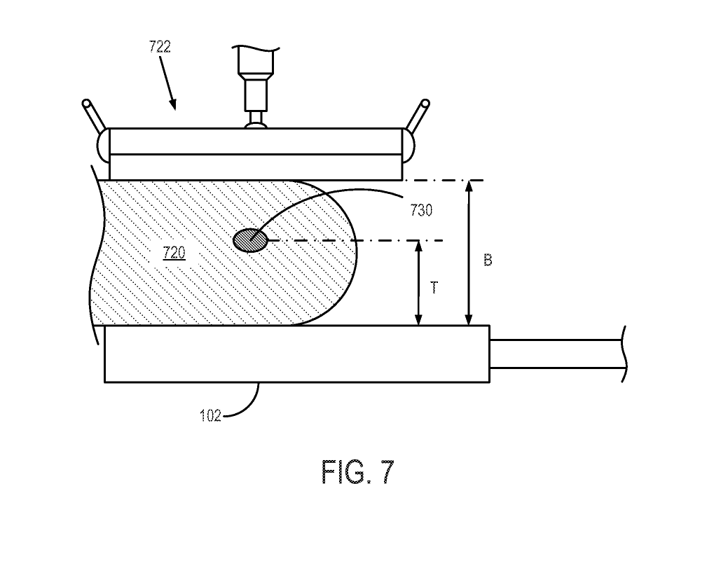

Referring now to FIGS. 6A and 6B, example of another configuration of an MBI system having a gantry-mounted upper detector head 102U and lower detector head 102L, which can be slidably moved relative to each other so as to provide access to the subject's breast for ultrasound imaging or biopsy, is illustrated. Like the configuration illustrated in FIG. 5, the configuration illustrated in FIGS. 6A and 6B include an adjustable coupling that permits movement of one detector head between a first and second position relative to another detector head. However, in the configuration illustrated in FIGS. 6A and 6B, this adjustable coupling includes support arms coupled to the support structure that allow movement of the detector heads along a direction that is coplanar with the respective imaging planes defined by the detector heads. FIG. 6A illustrates the MBI system in such a first position, and FIG. 6B illustrates the MBI system in such a second position. Like the MBI system configuration illustrated in FIG. 5, this MBI system configuration includes a support structure 602 having formed therein tracks 604. The support structure 602 is coupled to a gantry 606 that allows for the MBI system 600 to be positioned about the patient in a number of different orientations. Coupled to the tracks 604 are an upper support arm 608 and a lower support arm 610. The upper detector head 102U is coupled to the upper support arm 608, and the lower detector head 102L is coupled to the lower support arm 610. A motor (not shown in FIG. 6) drives the upper support arm 608 so that the upper detector head 102U is moved along a direction 612 towards or away from the lower detector head 102L.

The upper support arm 608 includes a retractable arm 614 that is coupled on one end to the upper detector head 102U and on the other to the upper support arm 608. Likewise, the lower support arm 610 includes a retractable arm 616 that is coupled on one end to the lower detector head 102L and on the other to the lower support arm 610. The retractable arms 614, 616 allow the upper detector head 102U and the lower detector head 102L to be slidably moved relative to each other. In the illustrated configuration, the upper detector head 102U may be moved along a longitudinal direction 622 towards or away from the support structure 602, and the lower detector head may be moved along a longitudinal direction 624 towards or away from the support structure 602. During an imaging procedure in which the physician desires to gain access to the breast 620, either for ultrasound imaging or to perform a biopsy, the upper detector head 102U may be moved towards the support structure 602 by retracting the retractable arm 614 within the upper support arm 608. At the same time, the lower detector head 102L may be moved away from the support structure 602 by extending the retractable arm 616 out of the lower support arm 610. A counterweight 618 is attached to the lower support arm 610 on the end opposite the lower detector head 102L so that when the lower detector head 102L is in its extended position, the MBI system 600 remains balanced and stable. In this MBI system configuration, the compression plate 212, including an acoustic coupling plate 210 or biopsy frame, may be coupled to the upper detector head 102U such that it is substantially parallel to the imaging plane defined by the upper detector head 102U, as illustrated in FIGS. 6A and 6B.

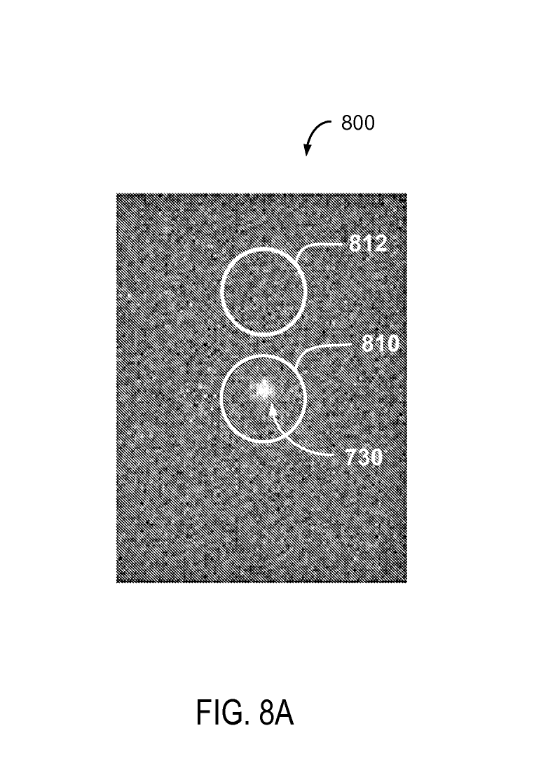

Referring now to FIG. 7, example of another configuration of a combined MBI—ultrasound system includes an ultrasound system, such as a wholebreast US (WBUS) system, 722 having an ultrasound paddle of dimensions similar to dimensions of the MBI detector head 102. Implementations of WBUS systems include, for example, a combined ultrasound probe and compression paddle device marketed under the trademark SomoVuTM (U-Systems, Sunnyvale, California). The ultrasound system 722 is normally designed to be placed directly on the breast tissue with the patient supine. The operator can then perform an automated scan of the breast. In the described configuration of the MBI system, the WBUS paddle acts as one part of a compression device to lightly compress breast tissue between the WBUS paddle and an MBI detector 102. This configuration of the MBI system also includes an MBI detector head 102 including a single gamma camera that can be positioned underneath the breast 720.

A related configuration of a combined MBI—ultrasound system includes the capability for elastography on the ultrasound system. Examples of systems include an ultrasound probe with elastography capability marketed under the trademark AixplorerTM (SuperSonic Imagine, Aix-en-Provence, France). The Aixplorer is normally designed to be placed directly on the breast tissue with the patient supine. In the described configuration of the MBI system, the Aixplorer probe is placed on top of the acoustic coupling plate 210, and shearwave elastography is performed over the region of abnormal uptake identified in the MBI images. This configuration of the MBI system also includes an MBI detector head 102 including a single gamma camera that can be positioned underneath the breast 720.

In use, the patient is seated and the breast lightly compressed by the WBUS paddle and MBI detector 102, in a similar orientation to mammography. Functional imaging of the breast is performed using the MBI system and simultaneously the WBUS system 722 can complete a sweep across the breast 720 to obtain 3D images of the breast tissue. Upon completion of both image acquisitions, the MBI and WBUS images are co-registered. Advantages of this configuration include reduced scan time due to the simultaneous acquisition of both the MBI and WBUS images, and reduced likelihood of motion artifact causing misregistration. This configuration does not provide depth information from the MBI image, and can instead provide only the in-plane location of a lesion for co-registration with the WBUS image. Thus, the lesion must also be visible on WBUS in order to determine exact location if ultrasound-guided biopsy is planned or desired.

In order to determine the uptake of radionuclide delivered to the tumor or lesion region of interest (ROI), one has to know the size of the tumor. When using the MBI modality for determination of the tumor size, the precision of such determination is reduced and, in practice, becomes unreliable when the size of the tumor falls below the value that is about twice the resolving power of the MBI system. Empirical data show that existing MBI systems may not provide reliable tumor size determination when small tumors (with dimensions on the order of a few millimeters, for example 4 to 10 millimeters) are being considered. It is in this case when the proposed dual-modality system of FIG. 7 allows to very precisely estimate the tumor size based on the ultrasound capability of the system to answer the question of radioactivity per unit volume in the ROI. One of the strengths of the ultrasound imaging modality is its ability to accurately measure the size and depth of a lesion—the two characteristics that are traditionally difficult to determine from conventional MBI imaging data.

An embodiment of the method of the invention, employing both the ultrasound and MBI capabilities of the system, enables the quantification of tumor uptake of radionuclide in a lesion seen on MBI images with the use of tumor activity (determined with photon counts on a gamma detector) and tumor depth and size measured with the ultrasound modality.

In further reference to FIG. 7, volume of tumor 730 is calculated from three values D₁, D₂, D₃ representing extent of the tumor as measured with the system 722 along the Cartesian coordinates, for example. Of these three(3) estimates of tumor dimension, D₁ and D₂ represent the extents of the tumor in the xy-plane (substantially parallel to the plane of the MBI detector), and D₃ represents tumor thickness (along an axis transverse to the plane of the MBI detector, such as z-axis). While knowledge of all three values of Ds are required for estimation of tumor volume, correction for the effects of gamma-ray attenuation on the apparent activity observed in the MBI image requires only knowledge of D₃. Assuming, in one example, an ellipsoidal shape of the lesion, the volume of the tumor V_{T} is estimated as

$${V}_{T} = \frac{4}{3} π \frac{{D}_{1}}{2} \frac{{D}_{2}}{2} \frac{{D}_{3}}{2} = \frac{1}{6} π {D}_{1} {D}_{2} {D}_{3} \qquad Eq . ( 1 )$$

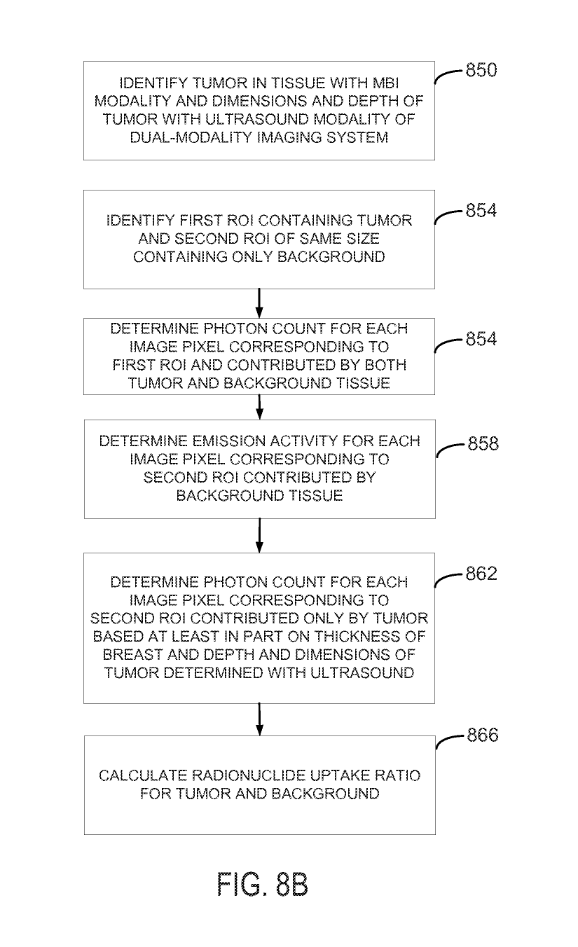

Determination of radionuclide uptake by the tumor tissue is now discussed in reference to FIGS. 8A, 8B and in further reference to FIG. 7. Once the tumor 730 has been identified in the breast tissue of thickness B in the image 800 obtained with the MBI portion of the dual MBI/ultrasound system of FIG. 7A, and its dimensions D₁, D₂, D₃ and depth T have been determined with the use of ultrasound modality of the system at step 850, two ROIs are chosen at step 854. These are a first ROI 810 fully containing the tumor 730 and a second ROI 812 (having substantially the same dimensions as those of the first ROI) that does not contain the tumor 730 and each pixel of which is located in the background portion of the image breast 720. Following the identification of the ROIs, the determination of photon counts in these ROIs is made, as shown in steps 858, 862 that generally can be performed in any order.

In particular, for any image pixel in the background ROI 812, photon emission representing radionuclide activity can be expressed at step 858 as

$${A}_{B} = \frac{{A}_{Bo}}{μ} [ 1 - exp ( - μ B ) ] = \frac{{A}_{Bo}}{μ} \cdot CB \qquad Eq . ( 3 )$$

where μ denotes the attenuation coefficient (in units of 1/cm) in soft tissue, for example for 140 keV gamma rays emitted by radionuclide such as Tc-99m. In comparison, for any pixel in the tumor-containing ROI 810, emission activity (photon count) represents the sum of radiant activity from both the tumor tissue itself and “background” activity corresponding to emission of the background tissue located in front of and behind the tumor tissue, and can be expressed at step 862 as

$${A}_{T + B} = \frac{{A}_{Bo}}{μ} {A}_{B} = \frac{{A}_{Bo}}{μ} [ 1 - exp ( - μ B ) ] + \frac{{A}_{To}}{μ} [ exp ( - μ ( T - \frac{D}{2} ) ) - exp ( - μ ( T + \frac{D}{2} ) ) ] + \frac{{A}_{Bo}}{μ} [ exp ( - μ ( T + \frac{D}{2} ) ) - exp ( - μ B ) ] \qquad Eq . ( 4 )$$

where for simplicity the term D₃ is now shown as D. Here, photons arrive at the detector from both the tumor and background tissue. After setting

$$\begin{matrix}{CB}_{1} = [ 1 - exp ( - μ ( T - \frac{D}{2} ) ] , & Eq . ( 5 A ) \\ CT = [ exp ( - μ ( T - \frac{D}{2} ) ) - exp ( - μ ( T + \frac{D}{2} ) ) ] , & Eq . ( 5 B ) \\ and & \\ {CB}_{2} = [ exp ( - μ ( T + \frac{D}{2} ) ) - exp ( - μ B ) ] , & Eq . ( 5 C ) \\ Eq . ( 4 ) can be re \text{-} written as & \\ {A}_{T + B} = \frac{{A}_{B 0}}{μ} [ {CB}_{1} + {CB}_{2} ] + \frac{{A}_{T 0}}{μ} CT & Eq . ( 6 )\end{matrix}$$

Taking into account the dependence for background activity A_{B0} from Eq. (3) into Eq. (5), the tumor activity contribution A_{TO} to the photon count of the chosen pixel in the first ROI 810 can be expressed, at step 866, as

$${A}_{T 0} = μ [ {A}_{T + B} - {A}_{B} \frac{{CB}_{1} + {CB}_{2}}{CB} ] \frac{1}{CT} \qquad Eq . ( 7 )$$

Summation of the A_{TO} over the tumor-containing ROI 810 at step 870 gives the total tumor-associated photon count inside the known tumor volume that has been defined by dimensions D₁, D₂, D₃ from the ultrasound measurement.

Accordingly, the ratio of the tumor to background activity (ratio of corresponding photon counts on the detector) can be calculated, if required, at step 874 as

$$\frac{T}{B} = [ {A}_{T + B} - {A}_{B} \frac{{CB}_{1} + {CB}_{2}}{CB} ] \frac{1}{CT} \frac{CB}{{A}_{B}} \qquad Eq . ( 8 )$$

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, it is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

The invention as recited in claims appended to this disclosure is intended to be assessed in light of the disclosure as a whole. The present invention has been described in terms of one or more preferred embodiments, and it should be appreciated that many equivalents, alternatives, variations, and modifications, aside from those expressly stated, are possible and within the scope of the invention.

What is claimed is:

1. A molecular breast imaging (MBI) system comprising:
a support structure having a surface dimensioned to support a breast in operation;
a first detector head operably coupled to the support structure and including:
a first gamma ray detector on the support structure and extending to define a first imaging plane;
a first collimator extending substantially coplanar with the first imaging plane in radiant communication with the first gamma ray detector;
an ultrasound paddle having an ultrasound transceiver therein, the ultrasound paddle attached to the support structure and rotatable about an axis to enable, in operation, compression of the breast between the ultrasound paddle and the first detector head ; and
a processor programmed to
acquire, from the first detector head, MBI imaging data representing an MBI image of a region of the breast containing a tumor tissue, the breast having received a radionuclide;
acquire, from the ultrasound transceiver, ultrasound imaging data representing an ultrasound image of the region of the breast to determine depth and size of the tumor tissue;
determine a first photon count corresponding to a first region of interest (ROI) from the MBI image, wherein the first ROI is defined to circumscribe an image of the tumor tissue;
determine a second photon count corresponding to a second ROI from the MBI image, the second photon count associated with a background portion of the MBI image that is devoid of the image of the tumor tissue;
calculate ratio of a first value of radionuclide uptake by the tumor tissue to a second value of radionuclide uptake by a background breast tissue that is devoid of tumor tissue based on the first and second photon counts, and depth and size of the tumor tissue
wherein the depth and size of the tumor tissue are obtained from the ultrasound imaging data.

2. An MBI system according to claim 1, further comprising an adjustable coupling between the first detector head and the support structure to permit movement of the first detector head between a first position with the first detector head substantially directly opposed to the ultrasound paddle and a second position with the first detector being not substantially directly opposed to the ultrasound paddle.

3. An MBI system according to claim 2, wherein the adjustable coupling includes a support art structured to slidably move the first detector head along a line belonging to the first imaging plane between the first and second positions.

4. An MBI system according to claim 1, wherein the first collimator includes a parallel-hole collimator section having a substantially circular outer periphery.

5. An MBI system according to claim 1, wherein the processor is programmed to calculate the ratio according to
$$\frac{T}{B} = [ {A}_{T + B} - {A}_{B} \frac{{CB}_{1} + {CB}_{2}}{CB} ] \frac{1}{CT} \frac{CB}{{A}_{B}} ,$$
wherein T is the value of radionuclide uptake by the tumor tissue, B is the value of radionuclide uptake by the background breast tissue, A_{T+B} is the first photon count, A_{B} is the second photon count,
wherein each of CB, CB_{'}, and CB₂ includes an exponential function of at least one of (i) the thickness of the tumor tissue in a direction substantially perpendicular to the first imaging plane, (ii) a thickness of the breast , and (iii) the depth of the tumor tissue, the thickness of the breast being determined from the ultrasound data.

6. A method for quantifying uptake of radionuclide by a breast of a subject, the method comprising:
in an imaging system having (i) a first MBI detector head operably coupled to the support structure and including a first gamma ray detector defining a first imaging plane, (ii) a first collimator extending substantially coplanar with the first imaging plane in radiant communication with the first gamma ray detector, and (iii) an ultrasound paddle having an ultrasound transceiver juxtaposed therewith and enabled to rotate about an axis,
compressing the breast that has received the radionuclide and that contains a tumor tissue between a first MBI detector head and the ultrasound paddle;
receiving, from the first MBI detector, first imaging data representing the breast and, from the ultrasound transceiver, second imaging data representing said breast;
identifying a first region of interest (ROI) in an MBI image corresponding to the first imaging data such that the first ROI fully includes an image of the tumor tissue, and a second ROI in the MBI image that corresponds to a portion of the MBI image that is devoid of the image of the tumor tissue, the second RIO being substantially co-extensive with the first ROI;
and
quantitatively determining the photon count representing radionuclide uptake by the tumor tissue as a function of (i) a first photon count received by the first detector head from the first ROI, (ii) a second photon count received by the first detector head from the second ROI, and (iii) depth and size of the tumor tissue in the breast,
wherein the first and second photon counts are associated with the first imaging data, and the depth and size of the tumor tissue are calculated from the second imaging data.

7. A method according to claim 6, wherein a ratio of the photon count representing radionuclide uptake by the tumor tissue to a photo is calculated according to
$$\frac{T}{B} = [ {A}_{T + B} - ABCB 1 + CB 2 CB 1 CTCBAB ,$$
wherein T is the value of radionuclide uptake by the tumor tissue, B is the value of radionuclide uptake by the background breast tissue, A_{T+B} is the first photon count, A_{B} is the second photon count, and
wherein each of CB, CB₁, and CB₂ includes an exponential function of at least one of (i) the thickness of the tumor tissue in a direction substantially perpendicular to the first imaging plane, (ii) a thickness of the breast , and (iii) the depth of the tumor tissue, the thickness of the breast being determined from the ultrasound data.

8. A method according to claim 6, wherein the compressing includes compressing the breast between the ultrasound paddle and the first MBI detector head enabled to slidably move between a first position with the first detector head substantially directly opposed to the ultrasound paddle and a second position with the first detector being not substantially directly opposed to the ultrasound paddle.

9. An article of manufacture, comprising
a computer processor operably cooperated with an imaging system that includes an MBI imaging unit and an ultrasound imaging unit spatially coordinated to compress a breast of a subject therebetween, the breast containing a tumor tissue and having received a radionuclide; and
a non-transitory tangible computer readable medium having computer readable product code thereon which, when loaded on a computer, enables an operation of the computer processor
to receive first imaging data from the MBI imaging unit and second imaging data from the ultrasound imaging unit;
to determine, from the first imaging data, a first photon count acquired by the MBI imaging unit from a first area of an MBI image that fully contains an image portion representing the tumor tissue and a second photon count acquired by the MBI imaging unit from a second are of the MBI image that is devoid of a portion of the image of the tumor tissue; and
to quantify an uptake of radionuclide by the tumor tissue per unit volume based on the first and second photon counts associated with the first imaging data, and a depth and dimensions of the tumor tissue in the breast that have been calculated from the second imaging data.
